(19) United States
(12) Patent Application Publication
(10) Pub. No.: US 2019/0274272 A1
(43) Pub. Date: Sep. 12, 2019

(54) UNIQUE NATTO SOYBEAN CULTIVAR

(71) Applicant: FTE Genetics, LLC, Oskaloosa, IA (US)

(72) Inventors: Kyle Olson, Oskaloosa, IA (US); Gerald Lorenzen, Oskaloosa, IA (US)

(21) Appl. No.: 16/297,353

(22) Filed: Mar. 8, 2019

Related U.S. Application Data
(60) Provisional application No. 62/640,857, filed on Mar. 9, 2018.

(51) Int. Cl. A01H 6/54; A01H 5/10

(52) U.S. Cl. CPC A01H 6/542; A01H 5/10

(57) ABSTRACT

A new soybean cultivar designated NQS 151, seeds of soybean cultivar NQS 151, plants of soybean cultivar NQS 151, plant parts of soybean cultivar NQS 151, methods for producing a soybean plant produced by crossing soybean cultivar NQS 151 with itself or with another soybean cultivar, and the creation of variants by mutagenesis or transformation of soybean cultivar NQS 151 are disclosed. Commercial commodity products from the seeds of soybean cultivar NQS 151, food products comprising these commercial commodity products, and methods for making the same are also disclosed.

### CROSS-REFERENCE TO RELATED APPLICATIONS DISCLOSURE

The application claims the benefit of U.S. Provisional Patent Application No. 62/640,857 filed Mar. 9, 2018, entitled “Unique Natto Soybean Cultivar”, which is hereby incorporated by reference in its entirety as if fully restated herein.

### BACKGROUND

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possesses the traits to meet the program goals. The goal is to combine in a single cultivar an improved combination of desirable traits from the parental germplasm. In the case of this inventive soybean cultivar, some of the important traits comprise increased seed physical uniformity, decreased seed size, increased seed roundness, increased yield, increased tolerance to iron chlorosis, increased resistance to soybean cyst nematode, and increased tolerance to phtophthora root rot.

Soybeans are of the Plantae kingdom; Fabales order; Fabaceae family; Glycine genus; and G. max species. Soybean has the binomial name Glycine max (L.). Soybeans were a crucial crop in East Asia starting between 7000 and 66000 BC in China, 5000 to 3000 BC in Japan, and 1000 BC in Korea. Soybeans are now a major crop in United States, China, Brazil, and Argentina.

Soybeans are primarily used as a source of food (especially protein) and oil. This use of soybeans as a source of protein is especially important as consumers become more aware of their protein consumption and become more cautious in consuming protein from animal sources. Soybeans are an excellent source of protein for food products desired by vegetarian and vegan customers.

Soybeans are also used in many non-fermented food products, including but not limited to soy milk and tofu. Soybeans are used in many fermented food products, including but not limited to natto, kongnaul, soy sauce, fermented bean paste, and tempeh.

Natto is a traditional Japanese food made from soybeans fermented with Bacillus subtilis var. natto. Some eat it as a breakfast food and others serve it with soy sauce, karashi mustard, and Japanese bunching onion. Natto has a very strong smell and flavor, as well as a slimy or slippery texture. Characteristics of soybeans used for natto comprise the seeds being extra small, very round and white, as well as able to have acceptable appearance, flavor, and texture (sensorial and mechanical) after fermination with bacteria “Bacillus Subtilis”. The preferred natto is made with very small, white, and round soybean seeds that develop the appropriate flavor and texture when fermented.

Kongnamul is a traditional Korean food made from fermented Soybean sprouts. Kongnamul is sold in packages in the grocery stores, as well sold with sauces or as part of meals (such as with rice or in soups). Soybean sprouts are cooked before eating because its strong fishy aroma when raw becomes a sweet and nutty flavor when cooked. Critical characteristics of soybeans to be used to make Korean sprouting soybeans (i.e., kongnamul) comprise small and uniform seed size, as well as appropriate flavor and texture after germination and fermentation. The preferred kongnamul is made with small, white, round soybean seeds that develop sprouts with an appropriate size, flavor and texture when germinated and fermented.

Each year farmers need to choose a soybean seed variety that will lead to a profitable crop. Therefore there is a need for a soybean cultivar with a) increased yield and maturity for the areas of production, b) an increased resistance to lodging, c) increased disease resistance, and d) improved seed character in terms of higher protein content, rounder seed shape, and greater seed size uniformity. Also, soybeans adaptive to the environment where they will be farmed, as well as modifying the physiological characteristics of the soybean plant and seed in order to make the plants more efficiently harvested and the seed more efficiently conditioned (i.e., cleaned) is desired.

### SUMMARY OF DISCLOSURE

The disclosure below uses different embodiments to teach the broader principles with respect to compositions, articles of manufacture, apparatuses, processes for using them and apparatuses, processes for making them, and products produced by the process of making, along with necessary intermediates. This Summary is provided to introduce the idea herein that a selection of concepts is presented in a simplified form as further described below. This Summary is not intended to identify key features or essential features of subject matter, nor is this Summary intended to be used to limit the scope of claimed subject matter. Additional aspects, features, and/or advantages of examples will be indicated in part in the description which follows and, in part, will be apparent from the description, or may be learned by practice of the disclosure.

This disclosure comprises a new and distinctive soybean cultivar, designated NQS 151; its seeds, plants, as well as the methods for producing the new soybean cultivar, the commercial commodity products from the seeds, and the food products comprising these commodity products. The disclosure below uses different embodiments to teach the broader principles with respect to articles of manufacture, processes for using the articles, and products produced by the process of making, along with necessary intermediates. This Summary is provided to introduce the idea herein that a selection of concepts is presented in a simplified form as further described below. This Summary is not intended to identify key features or essential features of subject matter, nor is this Summary intended to be used to limit the scope of claimed subject matter. Additional aspects, features, and/or advantages of examples will be indicated in part in the description which follows and, in part, will be apparent from the description, or may be learned by practice of the disclosure.

According to the disclosure, there is provided a new soybean cultivar designated NQS 151. The disclosure thus relates to the seeds of soybean cultivar NQS 151 (also designated as 8N146), to the plants of soybean cultivar NQS 151, to plant parts of soybean cultivar NQS 151, and to methods for producing a soybean plant produced by crossing soybean cultivar NQS 151 with itself or with another soybean cultivar, and the creation of variants by mutagenesis or transformation of soybean cultivar NQS 151. The disclosure also relates to the commercial commodity products from the seeds of soybean cultivar NQS 151, and the food products comprising these commercial commodity products.

Thus, any such methods using the soybean cultivar NQS 151 are part of this disclosure: selfing, backcrosses, hybrid production, crosses to populations, and the like. All plants produced using soybean cultivar NQS 151 as at least one parent are within the scope of this disclosure. This soybean cultivar can also be used in crosses with other, different, soybean plants to produce first generation (F₁) soybean hybrid seeds and plants with superior characteristics.

In another aspect, the present disclosure provides for single or multiple gene converted plants of soybean cultivar NQS 151. The transferred gene(s) may be a dominant or recessive allele. The transferred gene(s) should confer such traits as herbicide resistance, insect resistance, resistance for bacterial, fungal, or viral disease, male fertility, male sterility, enhanced nutritional quality (e.g., increased protein content), decreased seed size, size shape, yield, germination ability, and industrial usage. The gene may be a naturally occurring soybean gene or a gene introduced through genetic engineering techniques including, but not limited to, in vitro nucleic acid techniques, including recombinant deoxyribonucleic acid (DNA) and the direct injection of nucleic acid into cells or organelles; gene editing (CRISPr, TALEN, RNAi, or equivalent technologies); or fusion of cells beyond the taxonomic family that overcome natural physiological, reproductive, or recombination barriers and that are not techniques used in traditional breeding and selection.

In another aspect, the present disclosure provides regenerable cells for use in tissue culture of soybean plant NQS 151. The tissue culture will be capable of regenerating plants having the physiological and morphological characteristic of the foregoing soybean plant, and of regenerating plants having substantially the same genotype as the foregoing soybean plant. The regenerable cells in such tissue cultures can be embryos, protoplasts, meristematic cells, callus, pollen, leaves, anthers, roots, root tips, flowers, seeds, pods or stems. Still further, the present disclosure provides soybean plants regenerated from the tissue cultures of this disclosure.

The disclosure also relates to methods for producing a soybean plant containing in its genetic material one or more transgenes and to the transgenic soybean plants and plant parts produced by those methods. This disclosure also relates to soybean cultivars or breeding cultivars and plant parts derived from soybean cultivar NQS 151, to methods for producing other soybean cultivars, lines or plant parts derived from soybean cultivar NQS 151 and to the soybean plants, varieties, and their parts derived from use of those methods, including traditional breeding and genetic engineering. The disclosure further relates to hybrid soybean seeds, plants and plant parts produced by crossing soybean cultivar NQS 151 with another soybean cultivar.

Accordingly, some embodiments herein can, but need not always, be directed to a seed of soybean cultivar NQS 151, wherein a representative sample of the seed of said cultivar was deposited under ATCC Accession No. PTA-124920.

Similarly, some embodiments herein can, but need not always, be directed to a soybean plant, or a part thereof, wherein the soybean plant is produced by growing the cultivar.

Likewise, some embodiments herein can, but need not always, be directed to a tissue culture of regenerable cells produced from the soybean plant of the cultivar, such as wherein said regenerable cells of the tissue culture are produced from a plant part selected from the group consisting of leaf, pollen, embryo, cotyledon, hypocotyl, meristematic cell, root, root tip, pistil, anther, flower, stem and pod.

Also, some embodiments herein can, but need not always, be directed to a soybean plant regenerated from the tissue culture of the cultivar, such as wherein the soybean plant comprises morphological and physiological characteristics of cultivar NQS 151.

Additionally, some embodiments herein can, but need not always, be directed to soybean plant or a part thereof of the cultivar, such as wherein the soybean plant comprises some disease resistance to Iron chlorosis, Cyst nematode, Phytophthora root rot, and combinations thereof.

Further, some embodiments herein can, but need not always, be directed to a soybean plant of the cultivar, such as wherein the soybean plant comprises increased shatter resistance, decreased lodging, or combination thereof.

Also additionally, some embodiments herein can, but need not always, be directed to a soybean plant of the cultivar, such as wherein the soybean plant creates beans that comprise: an approximate round shape, pale in color, a water absorption test value greater than 0.8, a texture via mechanical measure of 0.28-0.31, a texture via sensory measure of less than 1.8, at least 98% sprouting rate in Warm Water Test, at least 95% sprouting rate in Five Day Sprouting Test, or combinations thereof.

Also further, some embodiments herein can, but need not always, be directed to a method for producing an F₁ hybrid soybean seed, such as wherein the method comprises crossing the soybean plant of the cultivar with a different soybean plant and harvesting at least one resultant F₁ hybrid soybean seed.

Some embodiments herein can, but need not always, be directed to hybrid soybean seed produced by such a method.

Additionally, some embodiments herein can, but need not always, be directed to a hybrid soybean plant, or a part thereof, produced by growing said hybrid soybean seed, such as is set forth herein.

Yet in addition, some embodiments herein can, but need not always, be directed to a method of producing a soybean plant with disease resistance, heat resistance, high protein content, high yield, or combinations thereof of the soybean plant of the cultivar or as disclosed herein, such as wherein the method comprises transforming the soybean plant as set forth herein with a transgene or a gene that confers disease resistance, heat resistance, high protein content, high yield or combinations thereof.

Yet also some embodiments herein can, but need not always, be directed to a soybean seed of the cultivar, such as a seed produced by crossing two soybean cultivars according to a single plant selection procedure of plant breeding to produce the soybean seed comprising at least one trait in Table 1 (below), wherein the process of single plant selection procedure comprises backcrossing until the at least one trait in Table 1 is dominant.

Yet further, some embodiments herein can, but need not always, be directed to a process of producing a soybean plant with increased disease resistance, increased shatter resistance, decreased lodging, and increased yield or combination thereof of soy seed of the cultivar or as disclosed herein, such as wherein the process of producing a soybean plant comprises crossing one soybean cultivar with another soybean cultivar according to single plant selection procedure of plant breeding and growing crossed seeds in maturity group 1 environment.

Yet further, some embodiments herein can, but need not always, be directed to a process of producing a soybean plant of the cultivar or as disclosed herein, such as wherein the process comprises plant breeding techniques, genetic engineering, or combinations thereof.

Yet in addition, some embodiments herein can, but need not always, be directed to a method of introducing a desired trait into soybean cultivar P NQS 151 or its progeny, such as wherein the method comprises:

(a) crossing a NQS 151 plant, representative seed having been deposited under ATCC Accession No. PTA-124920, with a plant of another soybean cultivar that comprises a desired trait to produce progeny plants wherein the desired trait is selected from the group consisting of herbicide resistance, insect resistance, increased protein content, increased yield, modified carbohydrate content, resistance to bacterial disease, resistance to fungal disease, increased shatter resistance, decreased lodging, or combinations thereof;
(b) selecting at least one progeny plant that comprises the desired trait to produce the selected progeny plant;
(c) crossing the selected progeny plant with the NQS 151 plant to produce a backcross progeny plant;
(d) selecting for the backcross progeny plant that comprises at least one desired trait, or physiological and morphological characteristic of soybean cultivar NQS 151 listed in Table 1 (below) to produce the selected backcross progeny plant; and
(e) repeating steps (c) and (d) three or more times in succession to produce a selected fourth or higher backcross progeny plant comprising the at least one desired trait and at least one physiological and morphological characteristics of soybean cultivar NQS 151 listed in Table 1.

Also some embodiments herein can, but need not always, be directed to a method of introducing a desired trait into soybean cultivar NQS 151, such as wherein the method involves plant breeding, genetic engineering, or combinations thereof.

Some embodiments herein can, but need not always, be directed to plant produced by a method such as set out herein, e.g., wherein the plant comprises at least one desired trait and at least one characteristic of soybean cultivar NQS 151 listed in Table 1 (below), yield listed in Table 2 (below), disease resistance listed in Table 3-5 (below), or combinations thereof.

Ever yet further, some embodiments herein can, but need not always, be directed to a process of producing a commodity plant product comprising: obtaining the soybean plant the cultivar or a part thereof; and producing the commodity plant product therefrom, such as wherein the commodity plant product is protein powder, protein concentrate, protein isolate, pea fiber, pea starch, pea meal, pea flour, pea hulls, or combinations thereof.

Yet ever additionally, some embodiments herein can, but need not always, be directed to food products comprising the commodity plant product of the cultivar or as disclosed herein.

Yet additionally, some embodiments herein can, but need not always, be directed to food products comprising at least one seed of the soybean cultivar.

### DETAILED DESCRIPTION

### Definitions

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

In the description and tables which follow, a number of terms are used. In order to provide a clear and consistent understanding of the specification and claims, including the scope to be given such terms, the following definitions are provided:

Allele: Allele is any or one of more alternative forms of a gene, all of which relate to one trait or characteristic. In a diploid cell or organism, the two alleles of a given gene occupy corresponding loci on a pair of homologous chromosomes.

Backcrossing: Backcrossing is a process in which a breeder repeatedly crosses hybrid progeny back to one of the parents, for example, a first generation hybrid F₁ with one of the parental genotypes of the F₁ hybrid.

Cotyledon: A cotyledon is a type of seed leaf. The cotyledon contains the food storage tissues of the seed.

Disease Resistance: Disease resistance genes comprise the ability to detect a pathogen attack and facilitate a counter attack again the pathogen.

Embryo. The embryo is the small plant contained within a mature seed.

Emergence: Emergence is the score that indicates the ability of the seed to emerge when planted 3″ deep in sand and with a controlled temperature of 25 C. The number of plants that emerge each day are counted. Based on this data, each genotype is given a 1 to 9 score based on its rate of emergence and percent of emergence. A score of 9 indicates an excellent rate and percent of emergence, and intermediate score of 5 indicates average ratings and a 1 score indicates a very poor rate and percentage of emergence.

Hilum: Hilum refers to the scar left on the seed which marks the place where the seed was attached to the pod prior to the seed being harvested.

Hypocotyl: A hypocotyl is the portion of an embryo or seedling between the cotyledons and the root. Therefore, it can be considered a transition zone between shoot and root.

Plant height: Plant height is taken from the top of the soil to the top node of the plant and is measured in centimeters.

Pod: Pod refers to the fruit of a soybean plant. It consist of the hull or shell (pericarp) and the soybean seeds.

Protein Percent: Soybean seeds contain a considerable amount of protein. Protein is generally measured by NIR spectrophotometry, and is reported on an as is percentage basis.

Quantitative Trait Loci (QTL): QTL refer to genetic loci that control to some degree numerically representable traits that are usually continuously distributed.

Regeneration: Regeneration refers to the development of a plant from tissue culture.

Shattering Resistance: Shatter resistance is the tendency of soybean pods to remain closed (i.e., sealed) and intact during and after maturity. The seal that keeps the soybean pod closed is intact and strong.

Seed Protein Peroxidase Activity: Seed protein peroxidase activity refers to a chemical taxonomic technique to separate cultivars based on the presence or absence of the peroxidase enzyme in the seed coat. There are two types of soybean cultivars: Those comprising high peroxidase activity (dark red color) and those comprising low peroxidase activity (no color).

Seeds Per Pound: Soybean sees vary in seed size, therefore, the number of seeds required to make up one pound also varies. This affects the pounds of seed required to plant a given area and can also impact end uses. Usually soybeans of the current disclosure were measured as weight per 100 seeds

Single Gene Converted (Conversion): Single gene converted (conversion) plant refers to plants which are developed by a plant breeding technique called backcrossing wherein essentially all of the desired morphological and physiological characteristic of a cultivar are removed in addition to the single gene transferred into the cultivar via the backcrossing technique or via genetic engineering.

Stem Vine Length: Stem vine length is measure in centimeters from the stem of the plant to the ground and is observed after flowering when pods are fully swollen.

Soybean cultivar NQS 151 (also designated as 8N146) of the current disclosure is a soybean with maturity group 1 and subgroup 4 area, which is bred for cultivation in Minnesota, South Dakota, and Nebraska local environmental conditions. Minnesota, South Dakota, and Nebraska are the currently most common environments for growing commercially available soybean cultivars.

The soybean cultivar NQS 151 has shown uniformity and stability with the plant and seed characteristics described in Table 1, the yield results for 2017 and 2018 in Table 2, the disease resistance results in Tables 3-5, the Natto Test results in Table 6, and the Bean Germination Test results in Table 7, as well as in the following cultivar information in this disclosure. The soybean cultivar NQS 151 has been self-pollinated a sufficient number of generations with careful attention to uniformity of plant type. The cultivar has been increased with continued observations for uniformity.

Table 1 has the morphologic and other characteristics of plant and seed of soybean cultivar NQS 151.

TABLE 1
Cultivar Description

| | NQS 151 |
|---|---|
| Plant type: | Soybean |
| Plant habit: | Semi-determinate |
| Seed coat color: | Yellow/white |
| Seed coat luster: | Dull |
| Seed size | 6-11 g/100 seeds |
| Hilum color: | Clear |
| Leaf color: | Dark green |
| Flower color: | White |
| Pod color: | Tan |
| Pubescene color: | Grey |
| Plant type: | Bushy |
| Plant height: | Tall |
| Plant growth type: | Semi-determinate |
| Plant habit: | Erect |
| Maturity group: | 1 |
| Maturity sub group | 4 |
| Herbicide reaction: | Susceptible to glyphosate |
| Iron chlorosis rating | Semi tolerant |
| Cyst nematode rating | Moderately resistant |
| Phytophthora root rot: | Semi tolerant |
| Yield (bushels per acre): | Greater than 60 |
| Seed shape: | Round |
| Germination test rate: | Greater than 90% |
| Natto test rate | Good or better |

The soybean variety chosen by a farmer is traditionally one that will grow well in the maturity group they farm in. A farmer has many varieties already commercially available to them. The seed cultivar of this disclosure has been successfully test-grown in several maturity groups. The seed cultivar of this disclosure can be grown in maturity group 1 area (e.g., Minnesota, South Dakota, and Nebraska), or other areas of similar environment. A major factor influencing maturity of all soybean plants is photoperiod. Breeders ideally want soybean seeds that are designed for (or will adapt to) the photoperiod of the latitude in which they are planted. The soybean plants need to flower and mature before they are killed by frost. Plant maturity is also dependent on other environmental factors, such as temperature, soil moisture, and indigenous plant diseases. The seeds of the cultivar of this disclosure was bred to thrive in the temperature, soil moisture (including but not limited to dry and sandy soil), and indigenous plant diseases of maturity group 1 area. The seeds of the cultivar of this disclosure can be planted in early May and be harvested in mid-October. Though, the seeds of this disclosure could also be planted earlier or later and still be successful depending on weather and soil conditions,

Soybean plants are annuals that are generally erect, bushy, and leafy plants that vary in height from 30 cm to 1 m. If given unlimited space, these plants would branch profusely, though traditional breeding has been done to produce varieties with short branches. Some traditional varieties produce determinate or indeterminate growth types. These types differ in their structure and in their preferred growing environment.

Determinate growth type plants have flowers borne in both axillary and terminal racemes, stem elongation ceasing with differentiation of the terminal bud. Determinate growth type soybean plants are adapted to a long growing season, in which the soybean plant can complete growth before flowering is initiated and still mature pods and seeds.

Indeterminate growth type plants have flowering beginning before stem elongation ceases and flowers are borne only on axillary racemes. These soybean plants are adapted to short growing seasons with flower and seed production proceeding before the soybean plant completes its growth.

The soybean cultivar of this disclosure is unique in that it has been bred to be semi-determinate, with a tall yet bushy structure, and a better yield in the maturity group 1 areas, that is Minnesota, South Dakota, and Nebraska.

Most soybean varieties have a tawny or gray colored pubescence on the stems, leaves, and pods. The number of seeds per pod may vary from one to three in most common commercial varieties. The soybean seed cultivar of this disclosure had a high yield, particularly because the soybean cultivar of this disclosure had 3-5 seeds per pod, and also has 2-3 more pods per plant than most common commercial soybean varieties. Commercially available soybean seed size varies from 5 to 55 g per 100 seeds, and can also have a range of shapes, usually oblong or oval. The soybean seed of this disclosure had uniformly small seeds (around 6-11 g per 100 seeds) that were uniformly round. While the seeds of commercially available soybeans varieties can vary from yellow, green, brown, black, or combinations of these colors, the seeds of the cultivar of this disclosure were roughly all pale yellow or white. The cultivar of this disclosure is small, uniformly round shape and pale color, can comprise good appearance when used in foods such as natto, and cheaper processing cost and final product cost. The more round the shape, and the more uniform in size the seeds are, the more efficient the conditioning (i.e., dry cleaning) and sorting of the seeds. The consistent size and shape of the soybean seeds allows for more efficient sorting of good seeds from lumps of dirt, rocks, or other debris, as well as from split and broken seeds. The more uniform the pale color of the seeds is, the more efficiently an optical eye can be used to aid in the sorting of good seed from bad seed and debris. The soybean flower is purple or white, or rarely, a combination of these two colors.

TABLE 2
Test A: Yield Test (Bushels/Acre)

| | Yield (Bushels/Acre) | | | | |
|---|---|---|---|---|---|
| | (2017) | (2017) | (2018) | (2018) | |
| | Repli- | Repli- | Repli- | Repli- | |
| Variety | cation 1 | cation 2 | cation 1 | cation 2 | Average |
| NQS 151 | 61.2 | 62.3 | 69.5 | 70.0 | 65.8 |
| 8N115 | 53.9 | 58.3 | 50.9 | 49.5 | 53.2 |
| 8N132 | 47.6 | 47.6 | 55.0 | 56.3 | 51.6 |
| 8N138 | 53.7 | 55.9 | 56.3 | 51.6 | 54.4 |
| 8N142 | 52.5 | 56.2 | 65.9 | 64.2 | 59.7 |
| Pioneer | 59.8 | 57.0 | 60.3 | 57.6 | |
| 91M10 | | | | | 58.7 |
| MN1011 | 58.3 | 56.9 | 61.7 | NA | 59.0 |

The yield values in Table 2 were the bushels/acre results of planting 300 seeds along a 16 foot row plot in maturity group 1 subgroup 4 area environmental conditions in 2017 and 2018.

Yield is a major decision factor for farmers choosing the seed cultivar they will use. Table 2 shows the Yield (bushels per acre) in replicates of several varieties including NQS 151 that are all grown in the same maturity group. Of the results in Table 2, NQS 151 has higher average and yearly yield test results than Pioneer 91M10 and Minnesota MN11011. NQS 151 also has higher average and yearly yield test results than several other varieties of soybean that that are commercially available from World Food Processing, LLC (Oskaloosa, Iowa).

The yield trial was set up in a randomized block design, two replicates per variety, rows were 16 feet in length and 1 row was harvested. Table 2 comprises variety Pioneer 91M10, which is a non-GMO variety considered to give a high yield. Pioneer 91M10 does not have the small round seeds preferred to make natto. Table 2 comprises variety Minnesota MN1011, which is a non-GMO variety that has a resistance to Cyst Nematodes. Pioneer 91M10 and MN1011 are feed bean varieties. Table 2 comprises several other commercially available natto bean varieties: 8N115, 8N132, 8N138, ad 8N142 sold by World Food Processing, LLC (Oskaloosa, Iowa).

Soybean cultivar NQS 151 embodiment of the current disclosure will stand erect without lodging. Lodging is the displacement of soybean plant stems from their proper and vertical placement. Amount of lodging is determined by the stockiness and strength of the plant stem. Lodging is dependent on plant structure and strength. Yield reductions of 20-25 percent may be caused by severe lodging due to the difficulty in harvesting soybeans from plants that are not in proper and vertical placement. The indeterminate soybean type plants are generally taller, with more slender stems; the determinate type varieties are shorter, stockier, and branch more heavily. The determinate soybean type plants are less likely to lodge on highly fertile soils than the indeterminate types. The seed cultivar of this disclosure grows into semi-determinate plants which are tall and strong, and as such, have a low incidence of lodging.

Soybean seeds of the cultivar of the current disclosure are produced in pods that tend to stay closed for a useful amount of time after they are ripe. As such, the soybean cultivar NQS 151 of the current disclosure has an acceptably low level of shattering (i.e., increased resistance to shattering) Shattering would have led to loss of seed before and during harvesting, and thus, a lower yield. Shattering can occur when the plant pods become dry, especially over long periods of time, such as in drought conditions. NQS 151 is bred to resist shattering under these dry conditions. NQS 151 of the current disclosure also has an extensive root system. This root system aids the soybean cultivar in getting moisture from the ground, in both wet and dry environments (e.g., drought conditions).

Another challenge of efficient commercial soybean growth and harvesting is the many diseases that can affect soybean plants. As illustrated in Table 1, the soybean seed cultivar of this disclosure comprises resistance to several diseases common to soybeans, especially soybeans grown in maturity group 1. Such diseases comprise iron deficiency chlorosis, cyst nematode, and phytophthora root rot.

TABLE 3
Soybean Iron Deficiency Chlorosis (IDC) Test Results

| Variety | Rating/Result |
|---|---|
| NQS 151 | Semi Tolerant: Slight yellow of plant tissue; |
| | Slight reduction in plant height on edges of |
| | areas where calcium carbonate has been |
| | deposited |
| 8N115 | Semi Susceptible: Moderate yellowing of plant |
| | tissue; Plant height reduced by 25% |
| 8N132 | Semi Susceptible: (above) |
| 8N138 | Semi Susceptible: (above) |
| 8N142 | Semi Tolerant: (above) |

The cultivar of the current disclosure (i.e., NQS 151) and several other cultivars commercially available from the World Food Processing LLC were evaluated for their susceptibility to iron deficiency chlorosis: Iron chlorosis (also called iron deficiency chlorosis: IDC) has been a challenge for soybean producers in various areas of Minnesota because of its soil conditions. The symptoms of IDC comprise distinctive yellow leaves with dark green veins as well as stunted growth. When severe, the leaves turn yellow or white and the outer edges turn brown as the plant cells die. The symptoms can be seen spotted across fields as the soil changes. Soils with high pH and soils with soluble salts can reduce the availability of iron for the plant. Most soils contain an abundance of iron, but its availability can be reduced by the pH and salts in the soil. Plant genetics has been found to be important to the soybean's ability to absorb iron, especially in soils not favorable for such.

TABLE 4
Soybean Cyst Nematode Rating/Test Results

| | NQS 151: 4 replications |
|---|---|
| 1. | Resistant |
| 2. | Moderately Resistant |
| 3. | Moderately Resistant |
| 4. | Moderately Resistant |

The results in for NQS 151 in Table 4 were based on the number of cysts on each soybean plant. Results were given as follows: Resistant: 0-14 cysts present; Moderately Resistant 15-42 cysts present; Moderately Susceptible 43-85 cysts present; Susceptible 86 or more cysts present. NQS 151 was shown to be at least moderately resistant.

Soybean cyst nematode, Heterodera glycines, has been found in most soybean growing areas around the world. SCN is a destructive pathogen with has led to more than a million dollars loss in the US. Because SCN can be present in fields without obvious surface symptoms, yield losses due to SCN can be much underestimated. SCN can cause soybean yield losses of more than 30%, especially in sandy and/or dry soil areas. The soybean cyst nematode is a roundworm that attacks the roots of a soybean plant. In central and northern Minnesota, the cyst nematode can complete three, or even four, life cycles including egg, juvenile, and adult development in a growing season. For this reason, any increase of resistance to soybean cyst nematode through genetics is of great interest to farmers.

TABLE 5
Phytophthora Root Rot Rating/Test Results

| Variety | Rating/Results |
|---|---|
| NQS 151 | Semi Tolerant |
| 8N115 | Semi Tolerant |
| 8N132 | Semi Susceptible |
| 8N138 | Semi Susceptible |
| 8N142 | Semi Tolerant |

Several cultivars (including NQS 151 and several commercially available cultivars) were evaluated as to their tolerance or susceptibility to phytophthora root rot The results were based on the following ratings:

Tolerant: No dark brown lesions on the stem; no yellowing or wilting of plant tissue. No stunting of plant growth

Semi Tolerant: Some yellowing of leaf tissue; small brown lesions observed on the lower portion of the plant reaching the 1^{st} of second node

Semi Susceptible: Yellowing and wilting of plant tissue; brown lesions observed from the lower portion of the plant to several nodes high

Susceptible: Plants are completely wilted and dead

Table 5 shows that NQS 151 was at least semi tolerant.

Phytophthora root rot (Phytophthora sojae) is of economic importance in fields with poor drainage, or areas with low-lying areas that are prone to flooding. Symptoms of phytophthora root rot comprise brown lesions, which can grow high on a soybean stem and girdle the stem, causing stunting or death of the plant. Phytophthora sojae can infect soybean plants at any stage of their growth. Varieties of soybean that are not fully susceptible could be stunted in growth, but not killed. Phytophthora sojae survives on crop material or soil in the form of oospores. When soil temperatures reach 69 F the oospores germinate and saturate the soil with zoospores. The zoospores are attracted to the soybean roots. Phytophthora root rot is of particular interest to farmers choosing a cultivar for their poorly drained soil, which is a common condition of acreage in maturity group 1. Phytophthora root rot is best managed by choosing cultivar with at least some resistance, such as the cultivar of this disclosure (i.e., NQS 151).

Consumers (and manufacturers) often prefer very small, white, and round soybean seeds, especially for use in consumer end products, such as natto and bean sprouts. The soybeans of the cultivar of this disclosure are bred to be very round, small, pale in color and uniform in appearance and they must have good product characteristics in natto and good germination characteristics for bean sprouts.

The seeds of the soybean cultivar NQS 151 of this disclosure were tested in natto (Test B), and found acceptable according to several characteristics, especially when compared to the results of other soybean cultivar varieties already commercially available by World Food Processing LLC. This test was one of the qualifying tests in evaluating the seeds of this cultivar. Natto consumption is very high in both Japan and in other countries were those of Japanese tradition reside, including the United States.

TABLE 6
Test B: Natto Test Results

| | Water | Stone | Texture | | Texture |
|---|---|---|---|---|---|
| | Absorption | Beans | Mechanical | Color | Sensory |
| | 2017/ | 2017/ | 2017/ | 2017/ | 2017/ |
| Variety | 2018 | 2018 | 2018 | 2018 | 2018 |
| NQS 151 | 3.0/3.2 | 0/0 | 0.309/0.281 | 1.5/1.3 | 1.7/1.5 |
| 8N115 | 1.1/1.2 | 0/1 | 0.379/0.394 | 3.0/3.4 | 3.2/3.5 |
| 8N132 | 2.4/2.8 | 0/0 | 0.326/0.290 | 1.7/1.4 | 2.0/1.9 |
| 8N138 | 2.0/2.1 | 0/0 | 0.377/0.369 | 2.3/2.6 | 3.0/3.3 |
| 8N142 | 2.3/2.6 | 0/0 | 0.369/0.322 | 2.0/1.8 | 2.5/2.0 |

A natto batch was made with each variety (grown in 2017 and grown in 2018) by soaking soybeans of each of the cultivars in Table 6 in water for about 18 hours. When the soybeans were done soaking, they were boiled or steamed in a pressure cooker. After cooking, the bacteria Bacillus subtilis was added to the cooked soybeans. The soybeans were then placed in a warm area to allow fermentation. When the fermentation process had reached its peak, the beans were allowed to cool and the end product (i.e., natto) was evaluated on five criteria:

1. Water absorption: Graded on 1-5 scale; 1 being the lowest amount of water absorbed by the soybeans, and 5 being the highest amount of water absorbed by soybeans. Acceptable: greater than 2.

2. Stone beans: Number of beans that did not absorb water. Any stone beans observed is a negative quality attribute. Acceptable: less than two.

3. Mechanical texture analysis: After the cooking process a sample of beans was put through a texture analyzer. Data was recorded in Newton's. The lower the Newton number, the softer the texture. Acceptable: less than 0.320.

4. Color: The color of the cooked soybeans was observed. 1-5 rating scale: 1 was a light brown color and 5 was a dark brown to black color. Acceptable: less than 2.

5. Sensory texture analysis: After the beans were fermented and cooled, the natto was analyzed for texture by taste testing. The natto was rated on a scale of 1-5: 1 being soft, almost mushy texture and 5 being hard almost crunchy texture. Acceptable: less than 2.

The seeds of the soybean cultivar NQS 151 of this disclosure were tested by Bean Germination Tests (Test C I: Warm Water Test; Test C II: Five Day Germination Test), and found to meet acceptable scores, especially when compared to the results of other soybean varieties already commercially available by World Food Processing LLC. These tests (Test C I and Test C II) were qualifying tests in evaluating the seeds of this cultivar. Bean sprouts (i.e., germinated soybeans) consumption is very high in both Korea and in other countries were those of Korean tradition reside, including the United States.

TABLE 7
Test C: Bean Germination Tests

| | | (I) Warm | (II) 5 Day | |
|---|---|---|---|---|
| Variety | | Water Test | Sprouting Test | Acceptable? |
| 1. | NQS151 | 99.00% | 95.50% | YES |
| 2. | 8N142 | 99.00% | 93.00% | YES |
| 3. | 8N156(LOT 185) | 93.00% | 86.00% | NO |
| 4. | 8N156(LOT138) | 93.00% | 94.00% | YES |
| 5. | 8N156(LOT 098) | 90.00% | 89.00% | NO |
| 6. | 8N156(LOT106) | 98.00% | 93.50% | YES |
| 7. | BYGLAND | 91.00% | 84.50% | NO |
| | 2012(LOT1) | | | |
| 8. | BYGLAND | 93.00% | 86.00% | NO |
| | 2012(LOT2) | | | |

The seeds of the soybean cultivar of this disclosure (i.e., NQS 151) were tested in Bean Germination Tests (Test C, parts I and II) and were found to create acceptable bean sprouts for foreign and domestic consumer markets. Table 7 comprises the results of the Warm Water Germination Test (Test C I) and the Five-Day Sprouting Germination Test (Test C II) for the soybean cultivar NQS 151 of this disclosure and several other soybean cultivars that are commercially available from World Food Processing LLC, as well as two lots of competitor soybean cultivar (i.e., Bygland 2012).

The Warm Water Germination Test consisted of placing 300 seeds in warm water, and after 20 minutes counting how many were cracked or broken. Results were recorded as percent of seeds not cracked or broken.

The Five-Day Sprouting Germination Test consisted of:
1. Placing 1400 seeds into a sprouting machine (that is, 4 trays with 350 seeds per tray). The base of each machine was filled with purified water.
2. Placing trays in complete darkness.
3. Changing water (should be purified water) once every 24 hours.
4. Watering the trays periodically for 5 days.
5. Controlling the temperature to maintain trays at 68-75 F.
6. Once 5 days are completed, evaluate seeds/germinated seeds for:

a) Sprout length longer than 7 cm, or be disqualified

Soybean heads should be free of cracking or breaking, or disqualified.

The soybean cultivar NQS151 of the current disclosure successfully passed the 90% or better germination rate for both Test C parts I and II, as shown in Table 7. For acceptable results, cultivars should meet at least 90% rate in both parts of Test C.

Choice of Breeding or Selection Methods:

Choice of breeding or selection methods depends on the mode of plant reproduction, the heritability of the trait(s) being improved, and the type of cultivar used commercially (e.g., F₁ hybrid cultivar, pure line cultivar, etc.). For highly heritable traits, a choice of superior individual plants evaluated at a single location will be effective, whereas for traits with low heritability, selection should be based on mean values obtained from replicated evaluations of families of related plants. Popular selection methods comprise pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program can comprise a periodic, objective evaluation of the efficiency of the breeding procedure. Evaluation criteria vary depending on the goal and objectives, but can comprise gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

These processes, which lead to the final step of marketing and distribution, usually take from 4 to 12 years from the time the first cross is made. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. Table 1 comprises the morphological and physiological characteristics of the soybean cultivar NQS 151 of this disclosure.

The goal of soybean plant breeding is to develop new, unique and superior soybean cultivars and hybrids. The breeder initially selects and crosses two or more parental lines, followed by repeated selfing and selection, producing many new genetic combinations. The breeder can theoretically generate billions of different genetic combinations via crossing, selfing and mutations. The breeder has no direct control at the cellular level.

Each year, the plant breeder selects the germplasm to advance to the next generation. This germplasm is grown under unique and different geographical, climatic and soil conditions, and further selections are then made, during and at the end of the growing season. The cultivars which are developed have some unpredictability. This unpredictability is because the breeder's selection occurs in unique environments, with no control at the DNA level (using conventional breeding procedures), and with millions of different possible genetic combinations being generated.

The soybean cultivar NQS 151 was bred with its superior morphological and physiological characteristics by careful selective breeding in maturity group 1 and subgroup 4 areas (which comprises the environment in Minnesota, South Dakota, and Nebraska). A breeder of ordinary skill in the art cannot predict the final resulting lines he develops, except possibly in a very gross and general fashion. This unpredictability results in the expenditure of large amounts of research monies, creativity, and diligence to develop superior new soybean cultivars.

An embodiment of the current disclosure comprises the development of the new soybean cultivar of this disclosure by the development and selection of soybean varieties, the crossing of these varieties, and the selection of superior hybrid crosses. The hybrid seed is produced by manual crosses between selected male-fertile parents or by using male sterility systems. These hybrids are selected for certain single gene traits such as pod color, flower color, pubescence color, plant physical characteristics, or disease resistance which indicate that the seed is truly a hybrid. Additional data on parental lines, as well as the phenotype of the hybrid, influence the breeder's decision whether to continue with the specific hybrid cross.

An embodiment of the current disclosure comprises pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which have commercial potential.

Pedigree breeding is used commonly for the improvement of self-pollinating crops. Two parents which possess favorable, complementary traits are crossed to produce an F₁. An F₂ population is produced by selfing one or several F₁s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

An embodiment of the current disclosure comprises the use of mass and recurrent selections to find and/or create the cultivar of the current disclosure (i.e., NQS 151 and its progeny). Mass and recurrent selections could be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

An embodiment of the current disclosure comprises the use of backcross breeding to find and/or create the cultivar of the current disclosure (i.e., NQS 151 and its progeny). Backcross breeding could be o transfer genes for a simply inherited, highly heritable trait into a desirable homozygous cultivar or inbred line which is the recurrent parent. The source of the trait to be transferred is called the donor parent. The resulting plant can comprise the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant can comprise the attributes of the recurrent parent (e.g., cultivar) and the desirable trait transferred from the donor parent.

An embodiment of the current disclosure comprises the use of single-seed descent procedure to find and/or create the cultivar of this disclosure (i.e., NQS 151 and its progeny). The single-seed descent procedure in the strict sense could refer to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

An embodiment of the current disclosure comprises the use of multiple-seed descent procedure to find and/or create the cultivar of this disclosure (i.e., NQS 151 and its progeny). In a multiple-seed procedure, soybean breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

An embodiment of the current disclosure comprises the use of mutation breeding techniques to find and/or create the cultivar of the current disclosure (i.e., NQS51 and its progeny). Mutation breeding could be another method of introducing new traits into soybean varieties to create the cultivar of the current disclosure. Mutations that occur spontaneously or are artificially induced can be useful sources of variability for a plant breeder. The goal of artificial mutagenesis is to increase the rate of mutation for a desired characteristic. Mutation rates can be increased by many different means comprising temperature, long-term seed storage, tissue culture conditions, radiation (such as X-rays, Gamma rays, neutrons, Beta radiation, or ultraviolet radiation), chemical mutagens (such as base analogues like 5-bromo-uracil), antibiotics, alkylating agents (such as sulfur mustards, nitrogen mustards, epoxides, ethylene amines, sulfates, sulfonates, sulfones, or lactones), azide, hydroxylamine, nitrous acid or acridness.

An embodiment of the current disclosure comprises the use of double haploids to find and/or create the cultivar of the current embodiment (i.e., NQS 151 and its progeny). The production of double haploids could also be used for the development of homozygous varieties in a breeding program. Double haploids would be produced by the doubling of a set of chromosomes from a heterozygous plant to produce a completely homozygous individual.

Proper testing should detect any major faults and establish the level of superiority or improvement over current cultivars. In addition to showing superior performance, the breeders of the current disclosure found a demand for a new cultivar that is compatible with industry standards or which creates a new market. The introduction of a new cultivar will incur additional costs to the seed producer, the grower, processor and consumer; for special advertising and marketing, altered seed and commercial production practices, and new product utilization. The testing preceding release of a new cultivar should take into consideration research and development costs as well as technical superiority of the final new cultivar of the current disclosure. As it is a seed-propagated cultivar, the new cultivar had to be feasible to produce seed easily and economically.

Based on knowledge of soil and growing conditions, as well as knowledge of cultivar characteristics potentially available in breeding stock, along with the time and patience for repeated selfing and selection (with or without mutation breeding processes), the breeders of the cultivar of the current disclosure were able to develop a soybean cultivar (i.e., NQS 151) with high protein content, high yield, uniform and round seeds, reduced lodging, reduced shattering, increased disease resistance.

An embodiment of the current disclosure comprises single or multiple gene converted plants of soybean cultivar NQS 151. The transferred gene(s) may be a dominant or recessive allele. The transferred gene(s) can confer such traits as high protein content, round seed shape, light seed color, structure characteristics so as to reduce lodging and to reduce shattering, and disease resistance. The gene may be a naturally occurring soybean gene or a gene introduced through genetic engineering techniques.

An embodiment of the current disclosure comprises regenerable cells for use in tissue culture of soybean cultivar NQS 151. The tissue culture can have a capability of regenerating plants comprising the physiological and morphological characteristics of the foregoing soybean plant, or of regenerating plants comprising substantially the same genotype as the foregoing soybean plant. The regenerable cells in such tissue cultures can be embryos, protoplasts, meristematic cells, callus, pollen, leaves, anthers, roots, root tips, flowers, seeds, pods, or stems. Still further, the present disclosure provides soybean plants regenerated from the tissue cultures of this disclosure.

Embodiments of the current disclosure comprise all means of breeding to create soybean cultivar NQS 151 and any progeny of soybean cultivar NQS 151. Choice of breeding or selection methods depends on the mode of plant reproduction, the heritability of the trait(s) being improved, and the type of cultivar used commercially (e.g., F₁ hybrid cultivar, pure line cultivar, etc.). For highly heritable traits, a choice of superior individual plants evaluated at a single location will be effective, whereas for traits with low heritability, selection should be based on mean values obtained from replicated evaluations of families of related plants. Popular selection methods can comprise pedigree selection, modified pedigree selection, mass selection, and recurrent selection.

The embodiments of this disclosure are also directed to methods for producing a soybean plant by crossing a first parent soybean plant with a second parent soybean plant, wherein the first or second soybean plant is the soybean plant for the soybean cultivar NQS 151 of the current disclosure. Further, both first and second parent soybean plants may be from the soybean cultivar NQS 151 of the current disclosure.

An embodiment of the current disclosure comprises methods for producing a soybean plant containing in its genetic material one or more transgenes of the cultivar of the current disclosure and to the transgenic soybean plants and plant parts produced by those methods. This disclosure also relates to soybean cultivars or breeding cultivars and plant parts derived from soybean cultivar NQS 151, to methods for producing other soybean cultivars, lines or plant parts derived from soybean cultivar NQS 151 and to the soybean plants, varieties, and their parts derived from use of those methods, comprising traditional breeding and genetic engineering. The disclosure further relates to hybrid soybean seeds, plants and plant parts produced by crossing soybean cultivar NQS 151 with another soybean cultivar.

Embodiments of this disclosure are also directed to methods for producing a soybean plant by crossing a first parent soybean plant with a second parent soybean plant, wherein the first or second soybean plant is the soybean plant for the soybean cultivar NQS 151. Further, both first and second parent soybean plants may be from soybean cultivar NQS 151. Therefore, any methods using soybean cultivar NQS 151 are part of this disclosure: selfing, backcrosses, hybrid breeding, and crosses to populations. Any plants produced using soybean cultivar NQS 151 as at least one parent are within the scope of this disclosure.

Methods comprise expression vectors introduced into plant tissues using a direct gene transfer method such as microprojectile-mediated delivery, DNA injection, electroporation and the like. Expression vectors can be introduced into plant tissues by using either microprojectile-mediated delivery with a biolistic device, of by using Agrobacterium-mediated transformation. Transformant plants obtained in the protoplasm of the disclosure are intended to be within the scope of this disclosure.

This disclosure is also directed to methods of using soybean cultivar NQS 151 to reproduce morphological and/or physical characteristics of NQS 151 in other soybean plants can comprise use of expression vectors introduced into plant tissues using a direct gene transfer method such as microprojectile-mediated delivery, DNA injection, electroporation and the like. Expression vectors are introduced into plant tissues by using either microprojectile-mediated delivery with a biolistic device, of by using Agrobacterium-mediated transformation. Transformant plants obtained in the protoplasm of the disclosure are intended to be within the scope of this disclosure.

An embodiment of the current disclosure comprises a method of introducing a desired trait into soybean cultivar NQS 151 or its progeny. The method can comprise the steps of: (a) crossing a NQS 151 plant, representative seed having been deposited under ATCC Accession No. PTA-124920, with a plant of another soybean cultivar that comprises a desired trait to produce progeny plants wherein the desired trait is selected from the group consisting of reduced lodging, increased yield, increased disease resistance, increased protein content, modified carbohydrate metabolism, and resistance to bacterial disease, fungal disease or viral disease, increased shatter resistance, or combinations thereof; (b) selecting one or more progeny plants that comprise the desired trait to produce selected progeny plants; (c) crossing the selected progeny plants with the NQS 151 plants to produce backcross progeny plants; (d) selecting for backcross progeny plants that comprise the desired trait and physiological and morphological characteristics of soybean cultivar NQS 151 listed in Table 1 to produce selected backcross progeny plants; and (e) repeating steps (c) and (d) three or more times in succession to produce selected fourth or higher backcross progeny plants that comprise the desired trait and all of the physiological and morphological characteristics of soybean cultivar NQS 151 listed in Table 1.

Another embodiment of the method for producing an F₁ hybrid soybean seed can comprise crossing the plant with a different soybean plant and harvesting at least one resultant F₁ hybrid soybean seed.

An embodiment of the process of producing a soybean plant can comprise producing a soybean plant comprising increased disease resistance, increased shatter resistance, reduced lodging, or combination thereof from a soybean seed, wherein the method comprises crossing soybean cultivar NQS 151 with another soybean cultivar according to single plant selection procedure of plant breeding and growing crossed seeds in dry or wet environments.

Another embodiment of the method of producing a soybean plant with disease resistance, high yield, high protein content, or combinations thereof of the soybean plant can comprise transforming the soybean plant with a transgene that confers disease resistance, high yield, high protein content, or combinations thereof.

An embodiment of the process of producing a soybean plant can comprise producing a soybean plant comprising increased disease resistance, decreased lodging, decreased shattering, increased protein content, increased yield, increased seed uniformity, pale color, and round shape or combinations thereof of soybean seed, wherein the soybean plant is genetically engineered.

Another embodiment of the present disclosure can comprise a soybean plant, or a part thereof, can comprise a plant or plant part, that is resistant to a disease selected from the group consisting of Iron Deficiency Chlorosis, Cyst Nematode (Heterodra glycines), Phytophthora Root Rot (Phytophthora sojae) and combinations thereof.

Another embodiment of the present disclosure can comprise a soybean plant comprising reduced lodging, reduced shattering, and a yield greater than 50 bushels/acre, or combinations thereof.

Another embodiment of the present disclosure can comprise a tissue culture of regenerable cells produced from a soybean plant of the current disclosure, wherein said cells of the tissue culture are produced from a plant part selected from the group consisting of leaf, pollen, embryo, cotyledon, hypocotyl, meristematic cell, root, root tip, pistil, anther, flower, stem and pod.

An embodiment of the current disclosure comprises the use of molecular biological techniques in the finding and/or creation of the soybean cultivar of this disclosure (i.e., NQS 151) and any progeny of that soybean cultivar.

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters or gene editing) in order to alter the traits of a plant in a specific manner. Such foreign additional and/or modified genes are referred to herein collectively as “transgenes”. Additionally, gene editing techniques enable the deletion, silencing, or increased expression of native genes within the genome, referred to herein as “gene editing”. Over the last twenty years several methods for producing transgenic and gene edited plants have been developed and the present disclosure, in particular embodiments, also relates to transformed versions of the claimed cultivar or line.

Plant transformation involves the construction of an expression vector which will function in plant cells. Such a vector comprises DNA comprising a gene under control of, or operatively linked to, a regulatory element (for example, a promoter). The expression vector(s) may be in the form of a plastid and can be used alone or in combination with other plasmids to provided transformed soybean plants using transformation methods to incorporate transgenes into the genetic material of the soybean plants(s).

Expression vectors can comprise at least one genetic marker operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and comprise, for example, genes that code for enzymes that metabolically detoxify a selective chemical agent which may be an antibiotic or an herbicide, or genes that encode an altered target which is insensitive to the inhibitor. A few positive selection methods are also known in the art.

Genes included in expression vectors should be driven by nucleotide sequence comprising a regulatory element, for example, a promoter. Several types of promoters are now well known in the transformation arts, as are other regulatory elements that can be used alone or in combination with promoters.

As used herein, “promoter” comprises reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control comprise promoters that initiate transcription in certain tissues, such as leaves, roots, seeds, fibers, xylem vessels, tracheids or sclerenchyma.

An inducible promoter is operably linked to a gene for expression in soybean. Optionally, the inducible promoter is operably linked to a nucleotide sequence encoding a signal sequence which is operably linked to a gene for expression in soybeans. An inducible promoter can be used in the current disclosure.

A constitutive promoter is operably linked to a gene for expression in soybean or the constitutive promoter is operably linked to a nucleotide sequence encoding a signal sequence with is operably linked to a gene for expression in soybean. Many different constructive promoters can be utilized in the current disclosure.

A tissue-specific promoter is operably linked to a gene for expression in soybean. Optionally, the tissue-specific promoter is operably linked to a nucleotide sequence encoding a signal sequence which is operably linked to a gene for expression in soybean. Plants transformed with a gene of interest operably linked to a tissue-specific promoter product the protein product of the transgene exclusively, or preferentially, in a specific tissue. Any tissue-specific or tissue-preferred promoter can be utilized in the current disclosure.

With transgenic plants according to the current disclosure, a foreign protein can be produced in commercial quantities. Thus, techniques for the selection and propagation of transformed plants, which are well understood in the art, yield a plurality of transgenic plants which can be harvested in conventional manner and a foreign protein then can be extracted from a tissue of interest or from total biomass. Protein extraction from plant biomass can be accomplished by known methods.

According to an embodiment of the current disclosure, the transgenic plant provided for commercial production of foreign protein is a soybean plant. In another embodiment, the biomass of interest is seed. For the relatively small number of transgenic plants that show higher levels of expression, a genetic map can be generated, primarily via conventional RFLP, PCR and SR analysis, which identified the approximate chromosomal location of the integrated DNA molecule. For additional methodologies, see Glick and Thompson, Methods in Plant Molecular Biology and Biotechnology, (CRC Press, Boca Raton) 269:284 (1993). Map information concerning chromosomal locations is useful for proprietary protection of a subject transgenic plant. If unauthorized propagation is undertaken and crosses made with other germplasm, the map of the integration region can be compared to similar maps for suspect plants, to determine if the latter comprise a common parentage with the subject plant. Map comparisons would involve hybridizations, RFLP, PCR, SSR and sequencing, all of which are conventional techniques.

Likewise, by means of the current disclosure, agronomic genes can be expressed in transformed or gene edited plants. An embodiment of the current disclosure comprises use of genetic engineering techniques to genetically engineer soybean plants to express various phenotypes of agronomic interest, such as increased soybean protein content, decreased lodging, decreased shattering, and/or increased disease resistance. Exemplary genes implicated in this regard comprise those categorized as genes that confer resistance to pests or disease, genes that confer resistance to an herbicide, genes that confer increased lodging resistance, shattering resistance, higher yield, higher protein content, and genes that confer or contribute to a value added trait (e.g., seed roundness, seed size, high protein content, lighter seed color).

As to genes that confer resistance to pests or disease: plant defenses are often activated by specific interaction between the product of a disease resistance gene (R) in the plant and the product of a corresponding avirulence (Ar) gene in the pathogen. A plant cultivar can be transformed with one or more cloned resistance genes to engineer plants that are resistant to specific pathogen strains. Engineered plants that contain these genes are intended to be within the scope of this disclosure.

As to genes that confer resistance to a herbicide—a herbicide that inhibits the growing point or meristem, such as an imidazolinone or a sulfonylurea, or a herbicide that inhibits photosynthesis. A plant cultivar can be transformed with one of more of cloned resistance genes to engineered plants that are resistant to specific herbicides. Engineered plants that contain these genes are intended to be within the scope of this disclosure.

As to genes that confer or contribute to a value-added trait, such as modified protein content, modified carbohydrate composition, and a transformation of a plant cultivar can comprise an increased protein content. Engineered plants that contain these genes that code (or confer) a value-added trait are intended to be within the scope of this disclosure.

Numerous methods for plant transformation have been developed, including biological and physical plant transformation protocols. The use of any of these transformations to find and/or create the soybean cultivar of the current disclosure (i.e., NQS 151 and its progeny) ae intended to be within the scope of this disclosure. See, for example, Miki et al., “Procedures for Introducing Foreign DNA into Plants” in Methods in Pant Molecular Biology and Biotechnology, Glick B. R. and Thompson, J. E. Eds. (CRC Press, Inc., Boca Raton, 1993) pages 67-88. In addition, expression vectors and invitro culture methods for plant cell or tissue transformation and regeneration of plants are available. See, for example, Glick B. R. and Thompson, J. E. Eds. (CRC Press, Inc., Boca Raton, 1993) pages 88-119.

Methods for Soybean Transformation:

A. Agrobacterium-mediated Transformation: One method for introducing an expression vector into plants based on the natural transformational system of Agrobacterium. See, for example, Moloney et al., Plant Cell Reports 8: 238 (1989).

B. Direct Gene Transfer: Several methods of plant transformation, collectively referred to as direct gene transfer, have been developed as an alternative to Agrobacterium-mediated transformation. A generally applicable method of plant transformation is microprojectile-mediated transformation wherein DNA is carried on the surface of microprojectiles measuring 1 to 4 um. The expression vector is introduced into plant tissues with a biolistic device that accelerates the microprojectiles to sufficient speed to penetrate plant cell walls and membranes. Another method for physical delivery of DNA to plants is sonication of target cells. Direct uptake of DNA into protoplasts using CaCl₂ precipitation, polyvinyl alcohol or poly-L-orthinine has also been reported.

C. Gene Editing: Several methods of gene editing including zinc finger nucleases (ZFNs), transcription activator-like effector nucleases (TALENs), and cluster regularly interspaced short palindromic repeats (CRISPR)-associated nuclease systems have been developed. Generally, once the ZFN, TALEN, or CRISPR constructs are introduced into and expressed in cells, the programmable DNA or RNA binding domain can specifically bind to a corresponding sequence and guide the chimeric nuclease to make a specific strand cleavage.

Following transformation of soybean target tissues, expression of the above-described selectable marker genes allows for preferential selection of transformed cells, tissues, and/or plants, using regeneration and selection methods now well known in the art.

The foregoing methods for transformation would typically be used for producing a transgenic cultivar. The transgenic cultivar could then be crossed with another (non-transformed or transformed) cultivar, in order to produce a new transgenic cultivar. Alternatively, a genetic trait which has been engineered into a particular soybean line using the foregoing transformation techniques could be moved into any of the line using traditional backcrossing techniques that are well known in the plant breeding arts. For example, a backcrossing approach could be used to move an engineered trait from a public, non-elite cultivar into an elite cultivar, or from a cultivar containing a foreign gene in its genome into a cultivar or cultivars which do not contain that gene. As used herein, “crossing” can refer to a simple X by Y cross, or the process of backcrossing depending on the context.

When the term soybean plant is used in the context of the present disclosure, it also comprises any single gene conversions of that cultivar. The term single gene converted plant as use herein refers to those soybean plants which are developed by a plant breeding technique called backcrossing wherein essentially all of the desired morphological and physiological characteristics of a cultivar are recovered in addition to the single gene transferred into the cultivar via the backcrossing technique. Backcrossing methods can be used with the present disclosure to improve or introduce a characteristic into the cultivar. The term backcrossing as used herein refers to the repeated crossing of a hybrid progeny back to the recurrent parent, i.e., crossing back 1, 2, 3, 4, 5, 6, 7, 8, 9, or more times to the recurrent parent. The parental soybean plant which contributes the gene for the desired characteristic is termed the nonrecurrent or donor parent. This terminology refers to the fact that the nonrecurrent parent is used one time in the backcross protocol and therefore does not recur. The parental soybean plant to which the gene or genes from the nonrecurrent parent are transferred is known as the recurrent parent as it is used from several rounds in the backcrossing protocol. In a typical backcross protocol, the original cultivar of interest (recurrent parent) is crossed to a second cultivar (nonrecurrent parent) that carries the single gene of interest to be transferred. The resulting progeny from this cross are then crossed again to the recurrent parent and the process is repeated until a soybean plant is obtained wherein essentially all of the desired morphological and physiological characteristics of the recurrent parent are recovered in the converted plant, in addition to the single transferred gene from the nonrecurrent parent as determined at the 5% significance level when grown in the same environmental conditions.

The selection of a suitable recurrent parent can be an important step for a successful backcrossing procedure. The goal of a backcross protocol is to alter or substitute a single trait or characteristic in the original cultivar. To accomplish this, a single gene of the recurrent cultivar is modified or substituted with the desired gene from the nonrecurrent parent, while retaining essentially all of the rest of the desired genetic, and therefore the desired physiological and morphological, constitution of the original cultivar. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new cultivar but that can be improved by backcrossing techniques. Single gene traits may or may not be transgenic; examples of these traits comprise male sterility, waxy starch, herbicide resistance, resistance for bacterial, fungal, or viral disease, insect resistance, male fertility, enhanced nutritional quality, industrial usage, yield stability and yield enhancement. These genes are generally inherited through the nucleus.

Further reproduction of the cultivar of the current disclosure can occur by tissue culture and regeneration, which is within the scope of the current disclosure. Tissue culture of various plant tissues and regeneration of plants therefrom is well known and widely published. Thus, another aspect of the current disclosure is to provide cells which, upon growth and differentiation, produce soybean plants comprising the physiological and morphological characteristic of soybean cultivar NQS 151.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, and the like. Means for preparing and maintaining plant tissue culture are well known in the art. By way of example, a tissue culture comprising organs has been used to produce regenerated plants.

An embodiment of the current disclosure also is directed to methods for producing the soybean cultivar NQS 151 by crossing a first parent soybean plant with a second parent soybean plant wherein the first or second parent soybean plant is a soybean plant of soybean cultivar NQS 151 of the current disclosure. Further, both first and second parent soybean plants can come from soybean cultivar NQS 151 of the current disclosure. Thus, any such methods using the soybean cultivar NQS 151 of the current disclosure are part of this disclosure: selfing, backcrosses, hybrid production, crosses to populations, and the like. All plants produced using soybean cultivar NQS 151 of the current disclosure as at least one parent are within the scope of this disclosure, including those developed from varieties derived from soybean cultivar NQS 151. This soybean cultivar NQS 151 could be used in crosses with other, different, soybean plants to produce first generation (F₁) soybean hybrid seeds and plants with superior characteristics. The cultivar of this disclosure can also be used for transformation where exogenous genes are introduced and expressed by the cultivar of the disclosure. Genetic variants created either through traditional breeding methods using soybean cultivar NQS 151 of the current disclosure or through transformation of soybean cultivar NQS 151 by any number of protocols known to those of skill in the art are intended to be within the scope of this disclosure.

The following describes breeding methods that may be used with soybean cultivar NQS 151 of the current disclosure in the development of further soybean plants, which are embodiments of the current disclosure. One such embodiment is a method for developing a soybean plant breeding program comprising: obtaining the soybean pant, or a part thereof, of soybean cultivar NQS 151 of the current disclosure utilizing said plant or plant part as a source of breeding material, and selecting a soybean cultivar NQS 151 progeny plant with molecular markers in common with soybean cultivar NQS 151 and/or with morphological and/or physiological characteristics selected from the characteristics listed in Table 1. Breeding steps that may be used in the soybean plant breeding program comprise pedigree breeding, backcrossing, mutation breeding, and recurrent selection. In conjunction with these steps, techniques such as RFLP-enhanced selection, genetic marker enhanced selection (for example SSR markers) and the making of double haploids may be utilized.

Another method of the current disclosure involves producing a population of soybean cultivar NQS 151 progeny soybean plants, comprising crossing filed pea cultivar NQS 151 with another soybean plant, thereby producing a population of soybean plants, which on average, derive 50% of their alleles from soybean cultivar NQS 151. A plant of this population may be selected and repeatedly selfed or sibbed with a soybean cultivar resulting from these successive filial generations. One embodiment of the current disclosure is the soybean cultivar produced by this method and that has obtained at least 50% of its alleles from soybean cultivar NQS 151.

One of ordinary skill in the art of plant breeding would know how to evaluate the traits of two plant varieties to determine if there is no significant difference between the two traits expressed by those varieties. Thus, the current disclosure comprises soybean cultivar NQS 151 progeny soybean plants comprising a combination of at least two NQS 151 traits selected from the group consisting of those listed in Table 1 through Table 6 as well as other traits mentioned in the current disclosure so that said progeny soybean plant is not significantly different for said traits than soybean cultivar NQS 151 as determined at the 5% significance level when grown in the same environmental conditions. Using techniques described herein, molecular markers may be used to identify said progeny plant as a NQS 151 progeny plant. Mean trait values may be used to determine whether trait differences are significant, and preferably the traits are measured in plants grown under the same environmental conditions.

Progeny of soybean cultivar NQS 151 of the current disclosure may also be characterized through their filial relationship with soybean cultivar NQS 151, as for example, being with a certain number of breeding crosses of soybean cultivar NQS 151. A breeding cross is a cross made to introduce new genetics into the progeny, and is distinguished from a cross, such as a self or a sib cross, made to select among existing genetic alleles. The lower the number of beading crosses in the pedigree, the closer the relationship between soybean cultivar NQS 151 of the current disclosure and its progeny. For example, progeny produced by the methods described herein may be within 1, 2, 3, 4, or 5 breeding crosses of soybean cultivar NQS 151.

As used herein, the term plant comprises plant cells, plant protoplasts, plant cell tissue cultures from which soybean plants can be regenerated, plant calli, plant clumps and plant cells that are intact in plants or parts of plants, such as embryos, pollen, ovules, flowers pods, leaves, roots, root tips, anthers, and the like.

In an embodiment of the current disclosure, the seed of soybean cultivar NQS 151, the plant produced from the seed, the hybrid soybean plant produced from the crossing of the cultivar with any other soybean plant, hybrid seed, and various parts of the hybrid soybean plant can be utilized as a commercial commodity, or to make a commercial commodity, as is or in the production of a human food, livestock food, or new material in industry. Such human food, livestock food, or new material could be comprised in whole or in part of matter from soybeans of the current disclosure (i.e., NQS 151 and its progeny). The matter could be from all parts of the soybean or from selected parts of the soybeans, such as protein, fiber, carbohydrate, ash, lip, or any combination thereof.

Further embodiments of the current disclosure comprise:

A. A soybean plant, or a part thereof, produced by growing the seed of soybean cultivar NQS 151, wherein a representative sample of seed of said cultivar was deposited under ATCC Accession No. PTA-124920, further comprising at least one transgene.

B. The soybean plant of A, wherein the at least one transgene confers upon the soybean plant resistance to bacterial disease, viral disease, or fungal disease, or combination thereof.

C. The soybean plant of A, wherein the at least one transgene confers upon the soybean plant drought tolerance or salt tolerance.

D. The soybean plant of A, wherein the at least one transgene confers upon the soybean plant shattering resistance and lodging resistance.

E. A method of producing soybean plant seed of pea cultivar NQS 151, comprising planting the seed of under conditions that result in the germination of the seed, growth of soybean plants and setting of progeny seed; and harvesting the progeny seed.

F. A method of producing a lodging and shatter resistance tolerant soybean plant, comprising: crossing a first soybean plant with at least one other soybean plant to produce progeny soybean plants, wherein the first soybean plant is the soybean plant of NQS 151; screening the progeny soybean plants to select a progeny soybean plant that is tolerant to drought conditions.

G. A method of producing a shatter resistant soybean plant, comprising: crossing a first soybean plant with at least one other soybean plant to produce progeny soybean plants, wherein the first soybean plant is the soybean plant NQS 151; screening the progeny soybean plants to select a progeny soybean plant that is resistant to shattering.

H. A method of producing a soybean plant with characteristics that will give the soybean cultivar a high yield (over 50 bushels/acre) and a high protein content, comprising: crossing a first soybean plant with at least one other soybean plant to produce progeny soybean plants, wherein the first soybean plant is the soybean plant NQS 151; screening the progeny soybean plants to select a progeny soybean plant that has the characteristics of high yield and high protein content.

I. A process of producing a commodity plant product comprising: obtaining the soybean plant of NQS 151 or a part thereof; and producing the commodity plant product therefrom.

J. The process of I, wherein the commodity plant product is protein powder, protein concentrate, protein isolate, pea fiber, pea starch, pea meal, pea flour, pea hulls, or combinations thereof.

K. The process of I, wherein the commodity plant product is used in making food products, comprising beverages, sauces, bakery, snacks, meat analogs, aerated desserts and confectionary, non-dairy milks, and combinations thereof. The list of food products above is not exhaustive.

L. A food product comprising the commodity plant product of I.

M. A food product of L further comprising proteins, starches, fibers, flours, or combinations thereof from non-soybean sources selected from the group consisting of beans, peas, chickpeas, sunflower seed, pumpkin, lentils, rice, oats, wheat, rye, tapioca, corn and combinations thereof.

N. The commodity plant product of I, wherein the commodity plant product is further modified by treatments selected from the group consisting of heating, milling, cooking, extruding, steaming, hydrolyzing, emulsifying, hydrogenating, acidifying, buffering, chemicalmodifying, and combinations thereof.

O. A food product comprising the soybean seed of claim 1, wherein the seed is in the form of natto.

P. A food product comprising the soybean seed of claim 1, wherein the seed is germinated into bean sprouts.

Q. A food product comprising the soybean seed of claim 1, wherein the food product is selected from the group comprising of natto, kongnaul, bean sprouts, soy sauce, fermented bean paste, tempeh, tofu, soy milk, meat analogs, nondairy cheese analogs, and combinations thereof.

Deposit Information

A deposit of the proprietary soybean cultivar designated NQS 151 disclosed above and recited in the appended claims has been made with the American Type Culture Collection (ATCC), 10801 University Boulevard, Manassas, Va. 20110. The date of deposit was Feb. 20, 2018. The deposit of 2500 seeds was taken from the same deposit maintained by GTE, since prior to the filing date of this application. All restrictions upon the deposit have been removed, and the deposit is intended to meet all of the requirements of 37 CFR § 1.801-1.809. The ATCC accession number is PTA-124920. The deposit will be maintained in the depository for a period of 30 years, or 5 years after the last request, or for the effective life of the patent, whichever is longer, and will be replace as necessary during that period.

In sum, it is important to recognize that this disclosure has been written as a thorough teaching rather than as a narrow dictate or disclaimer. Reference throughout this specification to “one embodiment”, “an embodiment”, or “a specific embodiment” means that a particular feature, structure, or characteristic described in connection with the embodiment is included in at least one embodiment and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment may be combined in any suitable manner with one or more other embodiments. It is to be understood that other variations and modifications of the embodiments described and illustrated herein are possible in light of the teachings herein and are to be considered as part of the spirit and scope of the present subject matter.

It will also be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/Figures should be considered only as exemplary, and not limiting, unless otherwise specifically noted. Furthermore, the term “or” as used herein is generally intended to mean “and/or” unless otherwise indicated. Combinations of components or steps will also be considered as being noted, where terminology is foreseen as rendering the ability to separate or combine is unclear.

As used in the description herein and throughout the claims that follow, “a”, “an”, and “the” comprises plural references unless the context clearly dictates otherwise. Also, as used in the description herein and throughout the claims that follow, the meaning of “in” comprises “in” and “on” unless the context clearly dictates otherwise. Variation from amounts specified in this teaching can be “about” or “substantially,” so as to accommodate tolerance for such as acceptable manufacturing tolerances.

The foregoing description of illustrated embodiments, including what is described in the Abstract and the Modes, and all disclosure and the implicated industrial applicability, are not intended to be exhaustive or to limit the subject matter to the precise forms disclosed herein. While specific embodiments of, and examples for, the subject matter are described herein for teaching-by-illustration purposes only, various equivalent modifications are possible within the spirit and scope of the present subject matter, as those skilled in the relevant art will recognize and appreciate. As indicated, these modifications may be made in light of the foregoing description of illustrated embodiments and are to be included, again, within the true spirit and scope of the subject matter disclosed herein.

In sum, it is important to recognize that this disclosure has been written as a thorough teaching rather than as a narrow dictate or disclaimer. Reference throughout this specification to “one embodiment”, “an embodiment”, or “a specific embodiment” means that a particular feature, structure, or characteristic described in connection with the embodiment is included in at least one embodiment and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment may be combined in any suitable manner with one or more other embodiments. It is to be understood that other variations and modifications of the embodiments described and illustrated herein are possible in light of the teachings herein and are to be considered as part of the spirit and scope of the present subject matter.

It will also be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/Figures should be considered only as exemplary, and not limiting, unless otherwise specifically noted. Furthermore, the term “or” as used herein is generally intended to mean “and/or” unless otherwise indicated. Combinations of components or steps will also be considered as being noted, where terminology is foreseen as rendering the ability to separate or combine is unclear.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise. Variation from amounts specified in this teaching can be “about” or “substantially,” so as to accommodate tolerance for such as acceptable manufacturing tolerances.

The foregoing description of illustrated embodiments, including what is described in the Abstract and the Modes, and all disclosure and the implicated industrial applicability, are not intended to be exhaustive or to limit the subject matter to the precise forms disclosed herein. While specific embodiments of, and examples for, the subject matter are described herein for teaching-by-illustration purposes only, various equivalent modifications are possible within the spirit and scope of the present subject matter, as those skilled in the relevant art will recognize and appreciate. As indicated, these modifications may be made in light of the foregoing description of illustrated embodiments and are to be included, again, within the true spirit and scope of the subject matter disclosed herein.

The compositions, articles, apparatuses, and methods of the present disclosure are capable of being incorporated in the form of a variety of embodiments, only a few of which have been illustrated and described. The disclosure may be embodied in other forms without departing from its spirit or essential characteristics. The described embodiments are to be considered in all respects only as illustrative and not restrictive, and the scope of the disclosure, therefore, is indicated by the appended claims rather than by the foregoing description. All changes which come within the meaning and range of equivalency of the claims are to be embraced within their scope.

Thus, although the foregoing disclosure has been described in some detail by way of illustration and example for purposes of clarity and understanding, it will be obvious that certain changes and modifications such as single gene modifications and mutations, somaclonal variants, variant individuals selected from large populations of the parts of the instant soybean cultivar and the like may be practiced within the scope of the disclosure, as limited only by the scope of the claims.

We claim:

1. A seed of soybean cultivar NQS 151, wherein a representative sample of the seed of said cultivar was deposited under ATCC Accession No. PTA-124920.

2. A soybean plant, or a part thereof, wherein the soybean plant is produced by growing the seed of claim 1.

3. A tissue culture of regenerable cells produced from the soybean plant of claim 2, wherein said regenerable cells of the tissue culture are produced from a plant part selected from the group consisting of leaf, pollen, embryo, cotyledon, hypocotyl, meristematic cell, root, root tip, pistil, anther, flower, stem and pod.

4. A soybean plant regenerated from the tissue culture of claim 3, wherein the soybean plant comprises morphological and physiological characteristics of cultivar NQS 151

5. A soybean plant or a part thereof of claim 2, wherein the soybean plant comprises some disease resistance to Iron chlorosis, Cyst nematode, Phytophthora root rot, and combinations thereof.

6. A soybean plant of claim 2, wherein the soybean plant comprises increased shatter resistance, decreased lodging, or combination thereof.

7. A soybean plant of claim 2, wherein the soybean plant creates beans that comprise: an approximate round shape, pale in color, a water absorption test value greater than 0.8, a texture via mechanical measure of 0.28-0.31, a texture via sensory measure of less than 1.8, at least 98% sprouting rate in Warm Water Test, at least 95% sprouting rate in Five Day Sprouting Test, or combinations thereof.

8. A method for producing an F₁ hybrid soybean seed, wherein the method comprises crossing the soybean plant of claim 2 with a different soybean plant and harvesting at least one resultant F₁ hybrid soybean seed.

9. A hybrid soybean seed produced by the method of claim 8.

10. A hybrid soybean plant, or a part thereof, produced by growing said hybrid soybean seed of claim 9.

11. A method of producing a soybean plant with disease resistance, heat resistance, high protein content, high yield, or combinations thereof of the soybean plant of claim 2, wherein the method comprises transforming the soybean plant of claim 2 with a transgene or a gene that confers disease resistance, heat resistance, high protein content, high yield or combinations thereof.

12. A soybean seed of claim 1, produced by crossing two soybean cultivars according to a single plant selection procedure of plant breeding to produce the soybean seed comprising at least one trait in Table 1, wherein the process of single plant selection procedure comprises backcrossing until the at least one trait in Table 1 is dominant.

13. A process of producing a soybean plant with increased disease resistance, increased shatter resistance, decreased lodging, and increased yield or combination thereof of soy seed in claim 1, wherein the process of producing a soybean plant comprises crossing one soybean cultivar with another soybean cultivar according to single plant selection procedure of plant breeding and growing crossed seeds in maturity group 1 environment.

14. A process of producing a soybean plant of claim 2, wherein the process comprises plant breeding techniques, genetic engineering, or combinations thereof.

15. A method of introducing a desired trait into soybean cultivar P NQS 151 or its progeny, wherein the method comprises:
(f) crossing a NQS 151 plant, representative seed having been deposited under ATCC Accession No. PTA-124920, with a plant of another soybean cultivar that comprises a desired trait to produce progeny plants wherein the desired trait is selected from the group consisting of herbicide resistance, insect resistance, increased protein content, increased yield, modified carbohydrate content, resistance to bacterial disease, resistance to fungal disease, increased shatter resistance, decreased lodging, or combinations thereof;
(g) selecting at least one progeny plant that comprises the desired trait to produce the selected progeny plant;
(h) crossing the selected progeny plant with the NQS 151 plant to produce a backcross progeny plant;
(i) selecting for the backcross progeny plant that comprises at least one desired trait, or physiological and morphological characteristic of soybean cultivar NQS 151 listed in Table 1 to produce the selected backcross progeny plant; and
(j) repeating steps (c) and (d) three or more times in succession to produce a selected fourth or higher backcross progeny plant comprising the at least one desired trait and at least one physiological and morphological characteristics of soybean cultivar NQS 151 listed in Table 1.

16. A method of introducing a desired trait into soybean cultivar NQS 151, wherein the method involves plant breeding, genetic engineering, or combinations thereof.

17. A plant produced by the method of claim 16, wherein the plant comprises at least one desired trait and at least one characteristic of soybean cultivar NQS 151 listed in Table 1, yield listed in Table 2, disease resistance listed in Table 3-5, or combinations thereof.

18. A process of producing a commodity plant product comprising: obtaining the soybean plant of claim 2 or a part thereof; and producing the commodity plant product therefrom, wherein the commodity plant product is protein powder, protein concentrate, protein isolate, pea fiber, pea starch, pea meal, pea flour, pea hulls, or combinations thereof.

19. Food products comprising the commodity plant product of claim 18.

20. Food products comprising at least one seed of the soybean cultivar of claim 1.
